(19) United States
(12) Patent Application Publication
(10) Pub. No.: US 2023/0139983 A1
(43) Pub. Date: May 4, 2023

(54) METHOD FOR OBTAINING COLLAGEN PEPTIDE FROM STARFISH, ELASTIC LIPOSOME COMPRISING STARFISH-DERIVED COLLAGEN PEPTIDE, AND COSMETIC COMPOSITION COMPRISING SAME

(71) Applicant: STARS TECH CO., LTD, Seoul (KR)

(72) Inventors: Seung Chan YANG, Seoul (KR); Ba Da WON, Seongnam-si (KR); Dong Hwee KIM, Seoul (KR); Seong Beom HAN, Seoul (KR)

(73) Assignee: STARS TECH CO., LTD, Seoul (KR)

(21) Appl. No.: 17/798,681

(22) PCT Filed: May 12, 2021
(86) PCT No.: PCT/KR2021/005972

§ 371 Date: Aug. 10, 2022

(30) Foreign Application Priority Data
May 19, 2020 (KR) 10-2020-0059674

(51) Int. Cl. C07K 1/12; A61K 8/14; A61K 8/65; A61Q 19/08; C07K 17/04

(52) U.S. Cl. CPC C07K 1/12; A61K 8/14; A61K 8/65; A61Q 19/08; C07K 17/04; A61K 2800/805

(57) ABSTRACT

A method for obtaining a collagen peptide from a starfish, an elastic liposome containing a starfish-derived collagen peptide, and a cosmetic composition containing the same are disclosed. Methods of using the collagen peptide or the elastic liposome are disclosed. The collagen peptide obtained from a starfish has excellent skin absorption rate and having anti-oxidation and wrinkle-improving activity. Thus, the collagen peptide may replace existing animal collagen to provide collagen with high extraction efficiency.

### TECHNICAL FIELD

The present invention relates to a method for obtaining a collagen peptide from a starfish, an elastic liposome comprising a starfish-derived collagen peptide, and a cosmetic composition comprising the same, and more specifically, to a method for obtaining a low molecular weight collagen peptide having anti-oxidation and skin wrinkle improvement effects from a starfish, an elastic liposome carrying the starfish-derived collagen peptide, and a cosmetic composition for improving skin wrinkles that has excellent skin absorption and excellent antioxidative effect by containing the same.

### BACKGROUND ART

Collagen is a fibrous protein found in most animals, in particular, mammals, and is a substance that occupies most of all connective tissues in the body, such as skin and cartilage. Collagen is shaped like a rope in which three polypeptide molecules are twisted into a triple helix.

Since collagen is involved in skin moisture content, it is commonly known that eating collagen-rich foods may prevent skin aging, joint weakness, and damage to blood vessels. However, since it is decomposed into amino acids such as glycine and proline through a proteolytic process and then absorbed during actual intake and oral administration, vitamin A, vitamin C and iron necessary for collagen synthesis must be additionally taken together in order to supplement the lack of collagen through intake.

In addition, there are products in the market that contain collagen molecules or fibers themselves as products applied to the skin; however, since protein is a polymer, it is highly likely that it will not penetrate the skin and thus will also not have a significant effect, and even if it is in the form of a low molecular weight, it is impossible to penetrate the stratum corneum except for pores and sweat glands which are less than 0.1% of the skin.

Materials for collagen production have been mainly supplied from livestock animals such as cattle and pigs until now, but for reasons that harmful issues due to the mad cow disease outbreak have recently emerged and animal collagen cannot enter the halal market for religious reasons, research using marine organisms as the materials is actively attempted.

For example, Korean Patent No. 10-1071338 describes a method for obtaining a collagen hydrolyzate from marine organisms such as the skins or scales of blowfish or sea bream, and Korean Patent Laid-Open No. 10-2006-0091350 describes a polymer scaffold for tissue engineering manufactured using collagen extracted from marine organisms.

However, marine collagen obtained from marine organisms has a problem in that the extraction amount is limited and extraction efficiency is also lower than that of animal collagen because extractable marine organisms are limited.

On the other hand, starfish inhabiting the coastal waters are marine wastes that require a budget of 400 to 500 million won as an annual treatment cost, and although they have high fertility and regenerative capacity, they adversely affect the ecosystem of marine organisms, reducing the yield of sea farms, and are only a problem for fishermen, and thus, how to utilize them as another resource is being studied. After these starfish are dried as they are, some are used to increase the harvest by sprinkling them on farmland, or some are used to manufacture calcium carbonate-based fertilizers, and recently, use as a snow removal agent has been proposed.

Therefore, if it is possible to prepare a collagen peptide having an excellent skin absorption rate using readily available starfish, it will be possible to provide a cosmetic composition effective for skin improvement while solving environmental problems.

### DISCLOSURE

### Technical Problem

In order to solve the problems of the prior art, it is an object of the present invention to provide a method for preparing a collagen peptide from a starfish.

It is another object of the present invention to provide an elastic liposome comprising the starfish-derived collagen peptide.

It is another object of the present invention to provide a cosmetic composition for anti-oxidation, comprising the starfish-derived collagen peptide.

It is another object of the present invention to provide a cosmetic composition for improving skin wrinkles, comprising the starfish-derived collagen peptide.

It is another object of the present invention to provide a cosmetic composition for anti-oxidation, comprising an elastic liposome comprising the starfish-derived collagen peptide.

It is another object of the present invention to provide a cosmetic composition for improving skin wrinkles, comprising an elastic liposome comprising the starfish-derived collagen peptide.

### Technical Solution

In order to achieve the objects as described above, the present invention provides a method for preparing a starfish-derived collagen peptide, comprising the steps of: (a) treating a starfish with an alkaline solution to remove non-collagen substances; (b) adding the starfish from which the non-collagen substances were removed, into an acid solution containing one or more acid compounds of tartaric acid, ascorbic acid and citric acid to extract collagen; (c) adding a proteolytic enzyme to the extracted collagen solution to hydrolyze the extracted collagen; and (d) isolating the collagen peptide from the solution.

In the present invention, the acid solution may contain 0.05 to 0.5% by weight of the acid compounds.

In the present invention, the enzyme may be one or more of subtilisin, pepsin, collagenase, and trypsin.

In the present invention, the collagen peptide may have a molecular weight of 1,550 to 1,700 Da.

The present invention also provides an elastic liposome comprising: a phospholipid layer comprising phospholipids and a surfactant; and a starfish-derived collagen peptide carried inside the phospholipid layer.

In the present invention, the starfish-derived collagen peptide may contain 30% or more of hydrophilic amino acids.

In the present invention, the surfactant may be a glucoside-based, sucrose-based or glyceryl-based surfactant.

In the present invention, the elastic liposome may have a particle size of 50 to 600 nm.

The present invention also provides a cosmetic composition for anti-oxidation, comprising the starfish-derived collagen peptide prepared by the method.

The present invention also provides a cosmetic composition for improving skin wrinkles, comprising the starfish-derived collagen peptide prepared by the method.

The present invention also provides a cosmetic composition for anti-oxidation, comprising an elastic liposome comprising the starfish-derived collagen peptide prepared by the method.

The present invention also provides a cosmetic composition for improving skin wrinkles, comprising an elastic liposome comprising the starfish-derived collagen peptide prepared by the method.

### Advantageous Effects

According to the present invention, a collagen peptide having excellent skin absorption rate and having anti-oxidation and wrinkle-improving activity is prepared using starfish which adversely affect the marine ecosystem and are difficult to treat, and thus, it may replace existing animal collagen to provide collagen with high extraction efficiency. In addition, since provided is a method for using the collagen peptides by carrying them in elastic liposomes, it is possible to overcome the limitation of low skin absorption rate of animal collagen and marine collagen, thereby greatly improving the transdermal absorption rate, and providing a cosmetic composition effective for anti-oxidation and skin wrinkle improvement using the same.

### DESCRIPTION OF DRAWINGS

FIG. 1 shows a cell live/dead image according to the type of enzyme in one experimental example of the present invention. In FIG. 1, (a) is a subtilisin/live image, (b) is a pepsin/live image, (c) is a C-0130/live image, (d) is a trypsin/live image, (e) is a subtilisin/dead image, (f) is a pepsin/dead image, (g) is a C-0130/dead image, and (h) is a trypsin/dead image.

### MODE FOR CARRYING OUT THE INVENTION

Hereinafter, specific embodiments of the present invention will be described in more detail. Unless defined otherwise, all technical and scientific terms used in the present specification have the same meaning as commonly understood by those of ordinary skill in the technical field to which the present invention pertains. In general, the nomenclature used in the present specification is those well known and commonly used in the art.

The present invention relates to a method for preparing a collagen peptide from a starfish, an elastic liposome comprising the starfish-derived collagen peptide prepared by the method, and a cosmetic composition for anti-oxidation and improving skin wrinkles comprising the same.

The muscle tissue of a starfish has elasticity that allows it to prey on shellfish 1.5 times the size of its own arm, and has a variety of physiological functions, such as the ability to regenerate tissues that grows damaged arms. In addition, it is estimated that these properties are closely related to collagen.

However, since the body wall of starfish is complexly composed of bone fragments (calcium carbonate), proteins, pigments, odor components, and the like, there are many differences from collagen extraction materials of terrestrial animals. Thus, when the known extraction methods such as acetic acid extraction method and pepsin extraction method are directly applied, effective extraction is difficult.

That is, there are the following problems: a large amount (20-30% by weight) of bone fragments (calcium carbonate) are present in the body wall of starfish, so it is necessary to clarify the conditions for removing non-collagen substances; when collagen is extracted by the known acetic acid extraction method or acid protease extraction method, calcium carbonate present in the body wall and acetic acid react to cause a neutralization reaction, so it is difficult to maintain optimal extraction conditions; and, when an excess of acid is used to adjust the pH, the ionic strength of the solution increases due to a large amount of calcium acetate generated as a result of the neutralization reaction, and collagen is precipitated and incorporated into the enzyme reaction residue, so the loss of collagen is large, thereby lowering economical efficiency.

In addition, the thermal denaturation temperature of collagen is 25° C. in starfish, which is relatively low compared to about 35-40° C. in warm-blooded animals, so it must be treated at a low temperature to avoid thermal denaturation.

The method for preparing a collagen peptide from a starfish of the present invention comprises the steps of: (a) treating a starfish with an alkaline solution to remove non-collagen substances; (b) adding the starfish from which the non-collagen substances were removed, into an acid solution to extract collagen; (c) adding a proteolytic enzyme to the extracted collagen solution to hydrolyze the extracted collagen; and (d) isolating the collagen peptide from the solution.

The starfish that may be used in the present invention are not particularly limited as long as they belong to the class Asteroidea within the phylum Echinodermata, and, for example, Asterias amurensis, Ophioplocus japonicus, Asterina pectinifera, Certonardoa semiregularis, Ophiothrix koreana, Solaster paxillatus, Culcita novaeguineae, Ophioplocus japonicus, Acanthaster planci, Ophiactis savignyi, Astropecten polyacanthus, Coscinasterias acutispina, Astropecten scoparius, Protoreaster nodosus, Ophiothela danae, Astropecten scorparius Valenciennes, and the like may be used.

In the method of the present invention, the starfish bone fragments may be obtained by first cutting the starfish into pieces and then treating them with an alkali solution to remove non-collagen substances.

Inactive ingredients other than collagen, such as protein, subcutaneous fat, odor-causing ingredients (amines, fatty acids, carbonyl compounds, sulfur compounds, and the like) and inorganic substances (calcium carbonate) are present in significant amounts in the body wall of starfish, and thus, such non-collagen substances may be removed through treatment with an alkali solution.

The alkali solution is not particularly limited as long as it is a mixed solution having a pH sufficient to isolate the inactive ingredients from the starfish, and, for example, may be a mixed solution having a pH in the range of 9 to 14 including an alkali compound and a solvent.

The alkali compound is not particularly limited as long as it is an alkali salt capable of adjusting the pH of the solution, and, for example, may include any one or two or more selected from sodium hydroxide, calcium hydroxide, potassium hydroxide, and the like, and sodium hydroxide is most preferred. The solvent is not limited, and, for example, water may be used.

The alkali solution may contain 1 to 20% by weight of the alkali compound.

In order to treat the starfish with alkali, the starfish may be cut into pieces and immersed in an alkali solution and then left for 12 to 48 hours.

When the alkali treatment is completed, bone fragments with collagen attached to the body wall of the starfish may be obtained. The obtained starfish bone fragments preferably have a yield of about 10 to 30% by weight of the weight of the initial starfish.

The obtained starfish bone fragments are added to an acid solution to extract collagen.

As the acid, an acid compound capable of adjusting the pH may be used, and tartaric acid, ascorbic acid, citric acid or the like may be used. In a preferred embodiment of the present invention, the acid compound may be a mixture of tartaric acid and ascorbic acid in a weight ratio of 10:1 to 1:10.

The acid solution may contain 0.05 to 0.5% by weight, more preferably 0.1 to 0.4% by weight of the acid compound. When the concentration of the acid solution is too low, the collagen extraction efficiency is too low, and the extraction efficiency increases as the concentration of the acid solution increases, but when the concentration of the acid solution exceeds about 0.25% by weight, the efficiency decreases again. Thus, it is preferable in terms of extraction efficiency to use the acid solution within a range not exceeding 0.5% by weight.

In the present invention, it is preferable to accelerate collagen extraction by performing ultrasonication after adding the bone fragments of the starfish to the acid solution. The ultrasonication may be performed at 10 to 100 kHz for 20 to 200 minutes, more preferably at 30 to 50 kHz for 40 to 80 minutes.

When the ultrasonication is completed, it may be left for 5 to 15 hours until the acid-base reaction is completed.

Next, a proteolytic enzyme is added to hydrolyze the extracted collagen into low molecular weight collagen peptides.

The starfish-derived collagen peptide prepared by the method of the present invention has a molecular weight of about 1,550 to 1,700 Da depending on the type of enzyme, which is lower than that of fish collagen (marine collagen) of about 1,900 Da or pig skin collagen of about 2,400 Da. Thus, it may be expected to be more advantageous for skin penetration.

As the enzyme, subtilisin, pepsin, collagenase, trypsin and the like may be used, and subtilisin may produce a collagen peptide having the lowest molecular weight and is most desirable in terms of wrinkle improvement performance.

In one example of the present invention, it was confirmed that when the collagen peptide was decomposed using subtilisin as an enzyme, a collagen peptide having the most excellent wrinkle improvement effect compared to other enzymes could be prepared.

The enzyme is preferably added in an amount of 0.01 to 1% by weight, more preferably 0.05 to 0.4% by weight, based on the weight of the starfish bone fragment.

The enzyme treatment temperature and time are not particularly limited as long as the proteolytic enzyme may sufficiently hydrolyze the starfish. For example, the hydrolysis temperature and time may range from 10 to 65° C. and from 1 to 10 hours, respectively. The hydrolysis temperature may be a temperature at which a proteolytic enzyme has high activity, which is known and thus may be appropriately adjusted depending on the type of the enzyme. For example, the hydrolysis temperature may range from 35 to 40° C. for trypsin.

When the enzymatic treatment is completed, the collagen peptide may be isolated. In this case, the isolation of the collagen peptide may be performed, for example, by centrifuging to remove salt and separating the supernatant, and then freeze-drying the supernatant to obtain the collagen peptide in powder form.

The prepared starfish-derived collagen peptide has a particle size of about 1 µm in a solvent, has no cytotoxicity, and has antioxidative activity. This is in contrast to that neither pig skin collagen nor fish collagen have antioxidative activity.

In addition, the starfish-derived collagen peptide of the present invention has anti-wrinkle activity. In one example of the present invention, it was confirmed that when the anti-wrinkle activity was compared through the inhibition rate of MMP-1 expression in cells, the starfish-derived collagen showed a 2-3 times higher inhibition rate of MMP-1 expression than the fish collagen and pig skin collagen.

The starfish-derived collagen peptide of the present invention itself may be used in a cosmetic composition for anti-oxidation and improving skin wrinkles, or may be used by being carried in elastic liposomes.

The elastic liposome carrying the starfish-derived collagen peptide according to the present invention may solve the problem of difficulty for the collagen peptide to pass through the intercellular lipid of the stratum corneum, thereby overcoming the limitation of skin absorption rate and securing optimal collagen peptide performance.

In particular, it was found in the present invention that the efficiency of carrying collagen peptides isolated from starfish in elastic liposomes may be significantly increased compared to those of pig collagen or fish collagen which are generally used in the prior art. This is thought to be because the starfish-derived collagen peptide contains a large amount of hydrophilic amino acids compared to pig skin or fish collagen. As shown in Table 1 below, the starfish-derived collagen peptide has a hydrophilic amino acid ratio of about 40%, which is about 1.5 times higher than that of pig skin or fish collagen containing about 25% of hydrophilic amino acids.

TABLE 1

| | Starfish | Nile tilapia | Pig skin |
|---|---|---|---|
| Alanine | 105.0 | 124.0 | 115.0 |
| Arginine | 94.0 | 51.0 | 48.0 |
| Aspartic acid | 78.0 | 46.0 | 44.0 |
| Cysteine | - | 3.0 | - |
| Glutamic acid | 106.0 | 76.0 | 72.0 |
| Glycine | 232.0 | 333.0 | 341.0 |
| Histidine | - | 8.0 | 5.0 |
| Isoleucine | 19.0 | 10.0 | 10.0 |
| Leucine | 16.0 | 23.0 | 22.0 |
| Lysine | 18.0 | 23.0 | 25.0 |
| Methionine | - | 2.0 | 6.0 |
| Phenylalanine | 4.0 | 14.0 | 1.0 |
| Proline | 108.0 | 119.0 | 123.0 |
| Serine | 40.0 | 35.0 | 33.0 |
| Threonine | 34.0 | 23.0 | 16.0 |
| Tyrosine | 7.0 | 3.0 | 1.0 |
| Valine | 28.0 | 19.0 | 22.0 |
| H-proline | 111.0 | 86.0 | 97.0 |
| Total | 1000 | 998 | 981 |
| Hydrophilic total | 377.0 | 265.0 | 244.0 |
| Hydrophilic % | 38% | 27% | 25% |

In this respect, the starfish-derived collagen peptide of the present invention may contain 30% or more, preferably 35% or more, and particularly 38% or more of hydrophilic amino acids. In one example of the present invention, it was confirmed that the starfish-derived collagen peptide containing about 40% of hydrophilic amino acids exhibited remarkably superior carrying efficiency of elastic liposomes compared to pig skin or fish collagen peptides.

Elastic liposomes have been proposed to compensate for several disadvantages of existing liposomes, such as low carrying efficiency, instability in formulation, low solubility of active ingredients, lipid oxidation, and hydrolysis potential, and may be prepared by adding a surfactant that imparts elasticity to phospholipids.

The elastic liposome according to the present invention is composed of a phospholipid layer comprising phospholipids and a surfactant, and a starfish-derived collagen peptide as a substance carried inside the phospholipid layer. The components not only contain phospholipids having a structure similar to that of skin cells, but also have excellent deformability due to increased elasticity, so that they may effectively penetrate and move between the keratinocytes, thereby having excellent transdermal absorption efficiency.

The phospholipids act as intercellular lipids to prevent the skin effective ingredients from escaping out of the skin, and at the same time act as semi-permeable membranes that draw in external moisture by osmotic function.

In the present invention, the phospholipid component may be a phospholipid generally used in the art, for example, has a fatty acid chain having 12 to 24 carbon atoms, and may include one or more of phosphatidylcholine, phosphatidylethanolamine, phosphatidylserine, phosphatidylglycerol, and phosphatidylinositol, but is not limited thereto. In the present invention, the phospholipid component is preferably phosphatidylcholine.

The surfactant is included for the purpose of improving the transdermal absorption rate by imparting elasticity to the interface of the liposome phospholipid layer. As the surfactant, a glucoside-based, a sucrose-based or a glyceryl-based surfactant may be used, and a glucoside-based surfactant is most preferred.

As the glucoside-based surfactant, cetearyl glucoside, decyl glucoside, coco glucoside, behenyl alcohol, arachidyl alcohol, arachidyl glucoside, C₁₀₋₂₀ alkyl glucoside and the like may be used, and cetearyl glucoside is most preferred.

As the sucrose-based surfactant, sucrose monostearate, sucrose distearate and sucrose tristearate may be used.

In addition, as the glyceryl-based surfactant, polyglyceryl-6 caprylate, polyglyceryl-4 caprate, polyglyceryl-3 methylglucose distearate and the like may be used.

In one example of the present invention, it was confirmed that when an elastic liposome was prepared using cetearyl glucoside, which is a glucoside-based surfactant, as a surfactant, its skin absorption rate was 3 to 5 times superior to that of other surfactants.

The phospholipid and surfactant may be mixed in a weight ratio of 3:1 to 20:1, more preferably in a weight ratio of 7:1 to 12:1.

In addition, the starfish-derived collagen peptide of the present invention may be included in an amount of 1 to 100% by weight based on the weight of the phospholipid layer comprising a mixture of phospholipids and surfactants, and is not particularly limited. From the experimental example of the present invention, it could be seen that the range of collagen peptides having the best skin absorption rate was different depending on the content of the phospholipid layer, and the skin absorption rate was the best in the case of comprising 1% by weight of the phospholipid layer and 0.1% by weight of the collagen peptide based on the weight of the solvent.

The elastic liposomes carrying the starfish-derived collagen peptide have a particle size of 50 to 600 nm, and most have a particle size of 100 to 200 nm. This is much smaller than the collagen peptide, which is about 1 µm in a solvent. In the experimental example of the present invention, as the phospholipid content increased, the particle size also showed a tendency to increase, which is judged to be because the thickness of the elastic liposome membrane is increased when a certain amount or more of phospholipid is added.

Collagen peptides having a particle size of about 1 µm in a solvent are hardly absorbed into the skin in the stratum corneum, but the elastic liposomes may have an excellent skin absorption rate due to a smaller particle size and elasticity.

The starfish-derived collagen peptide of the present invention and the elastic liposome comprising the same have excellent antioxidative activity and skin wrinkle improvement effect, and thus may be used in a cosmetic composition.

The elastic liposome may be included in an amount of 0.1 to 50% by weight based on the total weight of the cosmetic composition.

The cosmetic composition of the present invention may also be prepared in any formulations conventionally prepared in the art, and may be formulated into, for example, a solution, a suspension, an emulsion, a paste, a gel, a cream, a lotion, a powder, a soap, a surfactant-containing cleansing, an oil, a powder foundation, an emulsion foundation, a wax foundation, a spray, a mask pack, and the like, but is not limited thereto.

In addition, the cosmetic composition of the present invention may include cosmetics comprising various additives of different ingredients depending on the type of cosmetics, such as facial cleansing cosmetics, basic cosmetics, color cosmetics, hair care cosmetics, and functional cosmetics.

### EXAMPLES

Hereinafter, the present invention will be described in more detail through examples. It will be apparent to those skilled in the art that these examples are only for illustrating the present invention and the scope of the present invention is not to be construed as being limited to these examples.

### Preparative Example 1: Preparation of Starfish-Derived Collagen Peptides

1,000 g of starfish were immersed in 1 L of a 5 wt% sodium hydroxide solution, and then left for 24 hours to remove non-collagen substances and to obtain 200 g of bone fragments to which collagen is adhered.

A mixed acid compound of tartaric acid and ascorbic acid in a ratio of 1:1 was added to 50 mL of distilled water in an amount of 0.05, 0.25, 0.5, 1.0 and 2.5 wt% together with about 2 g of undried starfish bone fragments. Thereafter, ultrasonication was performed at 38 kHz for 1 hour, and then it was left for 10 hours or more to terminate the acid-base reaction.

Subtilisin, pepsin, collagenase (C-0130), collagenase (C-0130) buffer solution, trypsin and trypsin buffer solution were each added thereto as the enzyme in an amount of 0.1 wt% based on the weight of the starfish bone fragment to decompose a collagen peptide into a low molecular weight form.

Through a centrifuge, the salt generated from the acid/base reaction in the lower layer was removed, and the supernatant was separated. The separated supernatant was freeze-dried to obtain a collagen peptide in powder form.

### Experimental Example 1: Confirmation of Physical Properties According to the Collagen Peptide Extraction Process

### 1-1. Extraction Efficiency According to the Amount of Acid Added

The extraction efficiency of collagen peptides according to the amount of acid added is summarized in Table 2 below.

TABLE 2

| | Example 1 | Example 2 | Example 3 | Comparative Example 1 | Comparative Example 2 |
|---|---|---|---|---|---|
| Acid addition amount (% by weight) | 0.05 | 0.25 | 0.5 | 1.0 | 2.5 |
| Extraction efficiency (% by weight) | 2.30 | 13.34 | 19.15 | 28.02 | 54.22 |
| Yield excluding calcium ascorbate | 1.50 | 6.02 | 4.50 | -2.23 | -18.91 |

Since calcium ascorbate formed by the reaction of ascorbic acid and calcium carbonate, a component of bone fragments, is water-soluble, it is included in the extraction efficiency when the supernatant is freeze-dried after centrifugation. Thus, as a result of confirming the yield excluding calcium ascorbate that may be generated under the assumption that 100% of the added ascorbic acid has reacted, it was confirmed that the highest yield is obtained when 0.25 wt% of the acid is added, and as the amount of the acid added increases, the yield gradually decreases.

This is thought to be because calcium tartrate is preferentially formed over calcium ascorbate, and then calcium tartrate formed by the reaction of tartaric acid and calcium carbonate is removed in the lower layer during centrifugation.

### 1-2. Extraction Efficiency According to the Type of Enzyme

In order to find out the extraction efficiency according to the type of enzyme, the results of adding 6 types of enzymes to 0.05 wt% of the acid-treated samples are shown in Table 3 below.

TABLE 3

| | Example 3 | Example 4 | Example 5 | Example 6 | Example 7 | Example 8 |
|---|---|---|---|---|---|---|
| Enzyme | subtilisin | pepsin | C-0130 | trypsin | C-0130 buffer solution | trypsin buffer solution |
| Extraction efficiency (% by weight) | 3.8 | 3.6 | 2.3 | 3.8 | 2.5 | 3.8 |

Extraction efficiency was calculated based on the dry mass after freeze-drying of the collagen extract relative to the mass of the bone fragment to which collagen is adhered after alkali treatment, and TESCA for C-0130 and EDTA dissolved in PBS for trypsin were used as buffers, respectively.

It was confirmed that extraction efficiency showed almost the same trend for each enzyme except for collagenase C-0130, and there was no significant difference even in the buffer solution.

### 1-3. Molecular Weight of Collagen Peptides According to the Type of Enzyme

The molecular weight of collagen peptides according to the type of enzyme was confirmed through gel permeation chromatograph (GPC), and is shown in Table 4 below.

TABLE 4

| | Example 3 | Example 4 | Example 5 | Example 6 | Comparative Example 3 |
|---|---|---|---|---|---|
| Enzyme | subtilisin | pepsin | C-0130 | trypsin | X |
| Molecular weight (Da) | 1587 | 1650 | 1681 | 1699 | 2981 |

From the table above, it was confirmed that the collagen extract exists in the form of a low molecular weight peptide of about 1,700 Da due to the influence of the enzyme regardless of the type of enzyme, and in the case of subtilisin, the lowest molecular weight collagen peptide of 1,600 Da or less may be prepared.

### 1-4. Cytotoxicity Test According to the Type of Enzyme (MTT Assay)

In order to measure cell viability, human fibroblast HDF was cultured in a medium containing DMEM, 10% FBS, and 1% penicillin-streptomysin for 24 hours. Thereafter, it was cultured for additional 24 hours after replacing the medium with a medium containing each of enzyme-treated samples in Examples 3 to 6 at a concentration of 0.2 to 1.0 mg/mL, and it was cultured for additional 4 hours after adding MTT solution thereto.

After the medium was removed, the results of adding DMSO and measuring the absorbance at 560 nm are shown in Table 5 below.

TABLE 5

Cell viability (%)

| | | | | | |
|---|---|---|---|---|---|
| Concentration (mg/mL) | 0.2 | 0.4 | 0.6 | 0.8 | 1.0 |
| Subtilisin | 92 | 95 | 94 | 96 | 92 |
| Pepsin | 89 | 89 | 88 | 96 | 94 |
| C-0130 | 89 | 85 | 85 | 89 | 89 |
| Trypsin | 97 | 85 | 89 | 93 | 89 |

In the viability test for each concentration according to the type of enzyme, all showed a viability of 85% or more, and in the case of subtilisin, it was confirmed that there was little cytotoxicity because the viability was 92% or more.

### 1-5. Cytotoxicity Test According to the Type of Enzyme (Live/Dead Imaging)

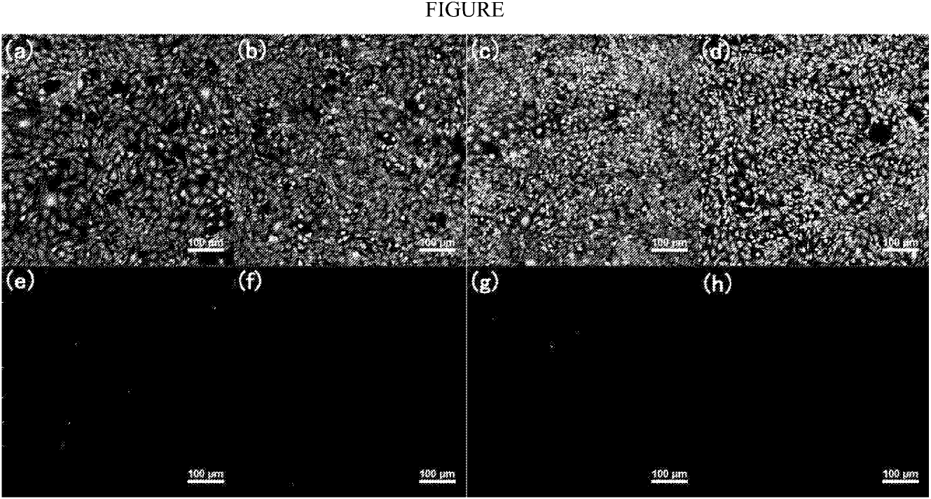

As in Experimental Example 1-4 above, after culturing at a concentration of 0.2 mg/mL for additional 24 hours, calcein-AM (live) and ethidium homodimer (dead) were added to the total PBS solution, and the results of imaging with confocal microscopy after 30 minutes are shown in FIG. 1.

In FIG. 1, (a) is a subtilisin/live image, (b) is a pepsin/live image, (c) is a C-0130/live image, (d) is a trypsin/live image, (e) is a subtilisin/dead image, (f) is a pepsin/dead image, (g) is a C-0130/dead image, and (h) is a trypsin/dead image.

It was confirmed that there was no cytotoxicity in all experimental groups.

### 1-6. Antioxidative Activity of Collagen Peptides According to the Type of Enzyme

The antioxidative activity of collagen peptides according to the enzyme was confirmed through the DPPH radical scavenging ability assay.

After the DPPH solution and the sample solutions of Examples 3 to 6 were reacted according to the concentration, the radical scavenging ability was confirmed through absorbance measurement at 517 nm, and the results are shown in Table 6 below. Antioxidative activity was confirmed by using vitamin C as a 100% control.

TABLE 6

Antioxidative activity (%)

| | | | | | |
|---|---|---|---|---|---|
| Concentration (mg/mL) | 0.2 | 0.4 | 0.6 | 0.8 | 1.0 |
| Vitamin C | 100 | 100 | 100 | 100 | 100 |
| Subtilisin | 93 | 90 | 89 | 86 | 82 |
| Pepsin | 89 | 89 | 88 | 84 | 86 |
| C-0130 | 89 | 85 | 85 | 82 | 83 |
| Trypsin | 94 | 90 | 89 | 88 | 88 |

Excellent antioxidative activity of about 90% or more was shown for all enzymes, and the most excellent antioxidative activity was shown at 0.2 mg/mL.

### 1-7. Comparison of Anti-Wrinkle Activity of Collagen Peptides According to Enzymes

Human fibroblast CCD-986sk was cultured in a medium containing DMEM, 10% FBS, and 1% penicillin-streptomysin for 24 hours. After replacing the medium with a medium containing each of enzyme-treated samples in Examples 3 to 6 at a concentration of 1 mg/mL, it was irradiated with UVB for 20 minutes and cultured for 24 hours. The culture supernatant was incubated with coating buffer, and then treated with washing buffer and blocking buffer. Thereafter, after primary and secondary antibody treatment at each diluted concentration, each culture supernatant was removed and treated with washing buffer. Finally, the results of incubating in the dark with pnPP (substrate solution) for 1 hour and measuring the absorbance at 405 nm were converted into the inhibition rate of MMP-1 expression and shown in Table 7 below.

TABLE 7

| Enzyme | Subtilisin | Pepsin | C-0130 | Trypsin |
|---|---|---|---|---|
| Inhibition rate of MMP-1 expression (%) | 62 | 44 | 32 | 51 |

From the table above, it was confirmed that the subtilisin-treated sample showed the best inhibition rate of MMP-1 expression compared to other enzymes.

### Experimental Example 2: Comparison of Starfish-Derived Collagen Peptide, Pig Skin Collagen Peptide, and Fish Collagen Peptide

An experiment was performed to compare the starfish-derived collagen peptides extracted according to the extraction process determined in Experimental Example 1 with the pig collagen peptides and the fish collagen peptides.

The pig collagen peptides and fish collagen peptides used in the experiment are shown in Table 8 below, respectively.

TABLE 8

| | Manufacturer | Raw material | Protein content |
|---|---|---|---|
| Pig skin collagen peptide | Xiamen Huaxuan Gelatin | Pig | ≥90% |
| Fish collagen peptide | Xiamen Huaxuan Gelatin | Nile tilapia | ≥90% |

### 2-1. Comparison of Cytotoxicity of Collagen Peptides

MTT assay cell viability test was performed in the same manner as in Experimental Example 1-4. As the starfish collagen, the collagen of Example 3 was used. MTT cell viability results are shown in Table 9 below.

TABLE 9

Cell viability (%)

| | | | | | |
|---|---|---|---|---|---|
| Concentration (mg/mL) | 0.2 | 0.4 | 0.6 | 0.8 | 1.0 |
| Starfish collagen peptide | 92 | 95 | 94 | 96 | 92 |
| Pig skin collagen peptide | 94 | 76 | 76 | 81 | 78 |
| Fish collagen | 86 | 78 | 88 | 79 | 90 |

From the table above, the pig skin collagen and fish collagen showed a cell growth rate of 80% or less at a concentration of 0.4 mg/mL or more, but overall, all three collagen samples did not show significant cytotoxicity.

### 2-2. Comparison of Molecular Weights of Collagen Peptides

The molecular weight of three types of collagen peptides was confirmed through gel permeation chromatograph (GPC), and is shown in Table 10 below.

TABLE 10

| Enzyme | Starfish collagen peptide | Pig skin collagen peptide | Fish collagen peptide |
|---|---|---|---|
| Molecular weight (Da) | 1587 | 2406 | 1901 |

It was confirmed that starfish-derived collagen has a much lower molecular weight than pig skin and fish collagen.

### 2-3. Comparison of Antioxidative Activity of Collagen Peptides

DPPH antioxidative activity was analyzed in the same manner as in Experimental Example 1-6 above, and is shown in Table 11 below.

TABLE 11

Antioxidative activity (%)

| | | | | | |
|---|---|---|---|---|---|
| Concentration (mg/mL) | 0.2 | 0.4 | 0.6 | 0.8 | 1.0 |
| Vitamin C | 100 | 100 | 100 | 100 | 100 |
| Starfish collagen peptide | 93 | 90 | 89 | 86 | 82 |
| Pig skin collagen peptide | -3 | -3 | -3 | -3 | -2 |
| Fish collagen peptide | -3 | -3 | -2 | -3 | -2 |

Starfish collagen showed excellent antioxidative activity, whereas both pig skin collagen and fish collagen did not show antioxidative activity.

### 2-4. Comparison of Anti-Wrinkle Activity of Collagen Peptides

The inhibition rate of MMP-1 expression was analyzed in the same manner as in Experimental Example 1-7 and shown in Table 12 below.

TABLE 12

| | Starfish collagen peptide | Pig skin collagen peptide | Fish collagen peptide |
|---|---|---|---|
| Inhibition rate of MMP-1 expression (%) | 62 | 54 | 23 |

As a result of comparing the anti-wrinkle activity through the inhibition rate of MMP-1 expression in cells by UV, it can be confirmed that the starfish-derived collagen peptide shows an inhibition rate of MMP-1 expression that is about 3 times higher than that of the fish collagen peptide and higher than that of the pig skin collagen peptide, thereby exhibiting remarkably excellent anti-wrinkle activity.

### Preparative Example 2: Preparation of Elastic Liposomes Carrying Collagen Peptides

Phospholipids, surfactants, and collagen peptides were placed in a 50 mL round flask according to the preparation ratio of Table 13 below and sufficiently dissolved in 20 mL ethanol. After the solvent was completely removed using a rotary evaporator, 20 mL of distilled water was added thereto to sufficiently dissolve the solvent. In order to homogenize the elastic liposome particles, ultrasonication was performed at 30 kHz for 15 minutes to prepare elastic liposomes.

TABLE 13

| Sample name | Phospholipid content (% by weight) | Surfactant content (% by weight) | Carried substance content (% by weight) |
|---|---|---|---|
| EL1/0.1 | 0.9 | 0.1 | 0.1 |
| EL3/0.1 | 2.7 | 0.3 | 0.1 |
| EL5/0.1 | 4.5 | 0.5 | 0.1 |
| EL10/0.1 | 9.0 | 1.0 | 0.1 |
| EL1/0.5 | 0.9 | 0.1 | 0.5 |
| EL3/0.5 | 2.7 | 0.3 | 0.5 |
| EL5/0.5 | 4.5 | 0.5 | 0.5 |
| EL10/0.5 | 9.0 | 1.0 | 0.5 |
| EL1/1 | 0.9 | 0.1 | 1.0 |
| EL3/1 | 2.7 | 0.3 | 1.0 |
| EL5/1 | 4.5 | 0.5 | 1.0 |
| EL10/1 | 9.0 | 1.0 | 1.0 |

### Experimental Example 3: Confirmation of Physical Properties of Elastic Liposomes

### 3-1. Carrying Efficiency of Elastic Liposomes

Using phosphatidylcholine as a phospholipid and polyglyceryl-6 caprylate and polyglyceryl-4 caprate (TEGO Solve 90, EVONIK) as surfactants, the carrying efficiency according to each composition ratio was measured.

After the preparation of elastic liposomes, the collagen peptides that were not carried were separated by filtration with a 450 nm syringe filter, and the purified elastic liposomes were crushed by ultracentrifuge, and then the carried collagen peptides were quantified by BCA assay. The carrying efficiency was calculated by calculating the ratio of quantitative values by BCA assay to total collagen peptides before carrying, and the results are shown in Table 14 below.

TABLE 14

| Sample name | Carrying efficiency (%) |
|---|---|
| EL1/0.1 | 88.3 |
| EL3/0.1 | 63.9 |
| EL5/0.1 | 27.9 |
| EL10/0.1 | 42.1 |
| EL1/0.5 | 57.3 |
| EL3/0.5 | 61.5 |
| EL5/0.5 | 55.1 |
| EL10/0.5 | 44.0 |
| EL1/1 | 31.7 |
| EL3/1 | 65.1 |
| EL5/1 | 53.2 |
| EL10/1 | 47.1 |

### 3-2. Comparison of Particle Sizes of Elastic Liposomes

The particle sizes of the starfish-derived collagen peptide and elastic liposomes were measured and shown in Table 15 below.

TABLE 15

| Sample name | Particle size (nm) |
|---|---|
| Starfish-derived collagen peptide | 1022 |
| EL1/0.1 | 109 |
| EL3/0.1 | 121 |
| EL5/0.1 | 129 |
| EL10/0.1 | 194 |
| EL1/0.5 | 200 |
| EL3/0.5 | 565 |
| EL5/0.5 | 388 |
| EL10/0.5 | 177 |
| EL1/1 | 145 |
| EL3/1 | 190 |
| EL5/1 | 204 |
| EL10/1 | 176 |

It can be confirmed that the starfish-derived collagen peptide showed a particle size of about 1 µm in the solvent and the elastic liposome particle size was in nm units that did not exceed 1 µm.

Overall, as the phospholipid content increased, the particle size also showed a tendency to increase, which is judged to be because the thickness of the elastic liposome membrane is increased when a certain amount or more of phospholipid is added.

### 3-3. Comparison of Skin Absorption Rate of Elastic Liposomes

In order to compare the skin absorption rate, after hydration of the skin layer of the acceptor plate coated with artificial skin, the samples were filled with buffer solution in each well of the donor plate. After the completion of hydration, the acceptor plate was filled with buffer and then placed on the donor plate for incubation. Thereafter, the results of analyzing the absorbance of each plate with a microplate reader and measuring the skin permeability are shown in Table 16 below.

TABLE 16

| Sample name | Skin absorption rate (mg/cm²/h) |
|---|---|
| Starfish-derived collagen peptide | 0 |
| EL1/0.1 | 2392 |
| EL3/0.1 | 1219 |
| EL5/0.1 | 642 |
| EL10/0.1 | 624 |
| EL1/0.5 | 1645 |
| EL3/0.5 | 968 |
| EL5/0.5 | 141 |
| EL10/0.5 | 1657 |
| EL1/1 | 740 |
| EL3/1 | 490 |
| EL5/1 | 0 |
| EL10/1 | 768 |

From the table above, in the case of collagen extract having a particle size of about 1 µm in a solvent, the skin absorption rate was not measured because the skin absorption was hardly made in the stratum corneum.

On the other hand, samples prepared with elastic liposomes showed various skin absorption rates depending on the ratio, which was somewhat similar to the particle size trend.

Therefore, it is preferable to prepare elastic liposomes with EL1/0.1, which has the carrying efficiency and particle size of the collagen extract in a suitable ratio and exhibits the best skin absorption rate, in consideration of process economics during mass production.

### 3-4. Analysis of Carrying Efficiency According to Surfactant

According to the preparation ratio of EL1/0.1, elastic liposomes were prepared with the following candidate surfactants, and carrying efficiency, particle size and skin absorption rate were compared. The prepared elastic liposome sample is named as follows according to the type of surfactant.

TABLE 17

| | | |
|---|---|---|
| Sample name | Surfactant type | Surfactant family and features |
| EL1/0.1-SF1 | TEGO SOLVE 90 | Polyglyceryl-6 caprylate (and) polyglyceryl-4 caprate |
| EL1/0.1-SF2 | TEGO CARE 450 | Polyglyceryl-3 methylglucose distearate |
| EL1/0.1-SF3 | TEGO CARE CG 90MB | Cetearyl glucoside |
| EL1/0.1-SF4 | TEGO CARE SE 121MB | Sucrose stearate |

The carrying efficiency of elastic liposomes according to the type of surfactant was measured in the same manner as in Experimental Example 3-1, and is shown in Table 18 below.

TABLE 18

| Sample name | Carrying efficiency (%) |
|---|---|
| EL1/0.1-SF1 | 82.0 |
| EL1/0.1-SF2 | 79.9 |
| EL1/0.1-SF3 | 88.3 |
| EL1/0.1-SF4 | 80.3 |

It was confirmed that the carrying efficiency of EL1/01-SF3 elastic liposomes using cetearyl glucoside surfactant was the highest.

### 3-5. Analysis of Particle Size According to Surfactant

The particle size of elastic liposomes according to the type of surfactant was measured and shown in Table 19 below.

TABLE 19

| Sample name | Particle size (nm) |
|---|---|
| EL1/0.1-SF1 | 109 |
| EL1/0.1-SF2 | 165 |
| EL1/0.1-SF3 | 110 |
| EL1/0.1-SF4 | 151 |

From the table above, it was confirmed to have a particle size in the range of approximately 100 nm without significant deviation depending on the type of surfactant.

### 3-6. Analysis of Skin Absorption Rate According to Surfactant

The skin absorption rate of elastic liposomes according to the type of surfactant was measured and shown in Table 20 below.

TABLE 20

| Sample name | Skin absorption rate (mg/cm²/h) |
|---|---|
| EL1/0.1-SF1 | 2392 |
| EL1/0.1-SF2 | 1333 |
| EL1/0.1-SF3 | 6455 |
| EL1/0.1-SF4 | 2070 |

From the table above, the skin absorption rate was generally about 2,000 mg/cm²/h, but the EL1/01-SF3 elastic liposome using cetearyl glucoside surfactant had a skin absorption rate of 6,455 mg/cm²/h, which exhibits a very high value of about 3 to 5 times compared to a sample using another surfactant.

### Experimental Example 4: Comparison of Elastic Liposomes Carrying Starfish-Derived Collagen Peptide, Pig Skin Collagen Peptide, and Fish Collagen Peptide

Using the same composition and surfactant as EL1/0.1-SF3 of Experimental Example 3, elastic liposomes carrying pig skin collagen peptide and fish collagen peptide, respectively, were prepared. The samples were named as shown in Table 21 below.

TABLE 21

| | |
|---|---|
| Sample name | Carried collagen peptide |
| EL-St | Starfish-derived collagen peptide |
| EL-Po | Pig skin collagen peptide |
| EL-Fi | Fish collagen peptide |

### 4-1. Carrying Efficiency of Elastic Liposomes According to Collagen Peptides

The carrying efficiency of elastic liposomes according to the type of collagen peptide was measured in the same manner as in Experimental Example 3-1, and is shown in Table 22 below.

TABLE 22

| Sample name | Carrying efficiency (%) |
|---|---|
| EL-St | 88.3 |
| EL-Po | 0 |
| EL-Fi | 17.0 |

It was confirmed that the carrying efficiency of the starfish-derived collagen peptide was more than 6 times higher. This is thought to be because the ratio of hydrophilic groups in the amino acid sequence of the starfish-derived collagen peptide is about 40%, which is higher than that of the pig skin and fish collagen peptides, so that the formation of elastic liposomes is made more easily.

### 4-2. Particle Size of Elastic Liposomes According to Collagen Peptides

The particle size of elastic liposomes according to the type of collagen peptide was measured and shown in Table 23 below.

TABLE 23

| Sample name | Particle size (nm) |
|---|---|
| EL-St | 110 |
| EL-Po | 98 |
| EL-Fi | 94 |

The particle size of the elastic liposomes showed a particle size of approximately 100 nm without significant deviation depending on the type of collagen peptide, but the particle sizes of EL-Po and EL-Fi were measured to be smaller. Judging from the carrying efficiency data, it is thought that elastic liposomes are prepared without carried substances, so the particle size is reduced.

### 4-3. Skin Absorption Rate of Elastic Liposomes According to Collagen Peptides

The skin absorption rate of elastic liposomes according to the type of collagen peptide was measured in the same manner as in Experimental Example 3-3, and is shown in Table 24 below.

TABLE 24

| Sample name | Skin absorption rate (mg/cm²/h) |
|---|---|
| EL-St | 6455 |
| EL-Po | 4466 |
| EL-Fi | 5428 |

It was confirmed that the skin absorption rate of the starfish-derived collagen peptides was very high compared to those of pig skin and fish collagen peptides.

### 4-4. Antioxidative Activity of Elastic Liposomes According to Collagen Peptides

The DPPH antioxidative activity of elastic liposomes according to the type of collagen peptide was measured and shown in Table 25 below.

TABLE 25

Antioxidative activity (%)

| | | | | | |
|---|---|---|---|---|---|
| Concentration (mg/mL) | 0.2 | 0.4 | 0.6 | 0.8 | 1.0 |
| Vitamin C | 100 | 100 | 100 | 100 | 100 |
| EL-St | 22 | 44 | 59 | 91 | 86 |
| EL-Po | 0 | -2 | -4 | -4 | -4 |
| EL-Fi | -1 | -5 | -3 | -5 | -5 |

As a result of the experiment, the elastic liposome carrying the starfish-derived collagen peptides exhibited excellent antioxidative activity, but the elastic liposomes carrying pig skin and fish collagen did not exhibit antioxidatve activity.

### 4-5. Inhibitory Activity of Skin Wrinkles of Elastic Liposomes According to Collagen Peptide

The inhibitory activity of skin wrinkles activity of elastic liposomes according to the type of collagen peptide was measured and shown in Table 26 below.

TABLE 26

| | EL-St | EL-Po | EL-Fi |
|---|---|---|---|
| Inhibition rate of MMP-1 expression (%) | 76 | 7 | 17 |

As a result of the experiment, it was confirmed that the elastic liposome carrying the starfish-derived collagen peptides showed excellent inhibitory activity of skin wrinkles, but the elastic liposomes carrying pig skin and fish collagen had no or weak inhibitory activity of skin wrinkles.

### Experimental Example 5: Confirmation of the Activity of Starfish-Derived Collagen Peptides According to the Type of Enzyme

The carrying efficiency and skin permeability of elastic liposomes of the starfish-derived collagen peptides obtained by using subtilisin, pepsin, C-0130 and trypsin as enzymes in Experimental Example 1 were measured in the same manner as in Experimental Examples 3 and 4, and then compared with the results for the pig skin collagen peptides and fish collagen peptides and are shown in Table 27 below.

TABLE 27

| | Starfish-derived collagen peptide (0.7 mg/mL) | | | | Pig skin collagen peptide (0.7 mg/mL) | Nile tilapia collagen peptide (0.7 mg/mL) |
|---|---|---|---|---|---|---|
| Enzyme | subtilisin | pepsin | C-0130 | trypsin | - | - |
| Extraction efficiency (% by weight) | 3.8 | 3.6 | 2.3 | 3.8 | - | - |
| Molecular weight (Da) | 1587 | 1650 | 1681 | 1699 | 2406 | 1901 |
| Cell viability (%) | 93.8 | 91.2 | 87.4 | 90.6 | 81 | 84.2 |
| Antioxidative activity (%) | 88 | 87.2 | 84.8 | 89.8 | -2 | -3 |
| Carrying efficiency (%) | 88.3 | 72 | 65.4 | 67.1 | 0 | 17 |
| Skin permeability (mg/cm²/h) | 6455 | 6127 | 5895 | 6273 | 4466 | 5438 |

From the table above, it could be confirmed that the case of using subtilisin as an enzyme had the lowest molecular weight, excellent cell viability, anti-wrinkle activity, carrying efficiency, and skin permeability. In addition, it can be confirmed that the starfish-derived collagen peptides extracted by pepsin, collagenase and trypsin still exhibit remarkably superior antioxidative activity, carrying efficiency and skin permeability compared to pig skin and Nile tilapia (fish) collagen peptides.

In particular, even in the case of C-0130 having the lowest carrying efficiency among the four enzymes, its carrying efficiency was about 3.8 times or more higher than that of the fish collagen peptide and its skin permeability was more excellent.

it is understood that this difference is not only due to the molecular weight according to the type of degradation enzyme, but also because the starfish-derived collagen peptide contains a large amount of hydrophilic amino acids compared to the pig skin or fish collagen peptide.

From the above description, those of ordinary skill in the technical art to which the present invention pertains will understand that the present invention may be implemented in other specific forms without changing the technical spirit or essential characteristics thereof. In this regard, it should be understood that the examples described above are illustrative and not restrictive in all respects. It should be construed that all changes or modifications derived from the meaning and scope of the claims to be described later rather than the detailed description and their equivalent concepts are included in the scope of the present invention.

1. A method for preparing a starfish-derived collagen peptide, comprising the steps of:
(a) treating a starfish with an alkaline solution to remove non-collagen substances;
(b) adding the starfish from which the non-collagen substances were removed, into an acid solution containing one or more acid compounds of tartaric acid, ascorbic acid and citric acid to extract collagen;
(c) adding a proteolytic enzyme to the extracted collagen solution to hydrolyze the extracted collagen; and
(d) isolating the collagen peptide from the solution.

2. The method for preparing a starfish-derived collagen peptide according to claim 1, wherein the acid solution contains 0.05 to 0.5% by weight of the acid compounds.

3. The method for preparing a starfish-derived collagen peptide according to claim 1, wherein, the enzyme is one or more of subtilisin, pepsin, collagenase, and trypsin.

4. The method for preparing a starfish-derived collagen peptide according to claim 1, wherein the collagen peptide has a molecular weight of 1,550 to 1,700 Da.

5. An elastic liposome comprising:
a phospholipid layer comprising phospholipids and a surfactant; and
a starfish-derived collagen peptide carried inside the phospholipid layer.

6. The elastic liposome according to claim 5, wherein the starfish-derived collagen peptide contains 30% or more of hydrophilic amino acids.

7. The elastic liposome according to claim 5, wherein the surfactant is a glucoside-based, sucrose-based or glyceryl-based surfactant.

8. The elastic liposome according to claim 5, wherein the elastic liposome has a particle size of 50 to 600 nm.

9. A cosmetic composition comprising the starfish-derived collagen peptide prepared by the method of claim 1.

10. A method for improving skin wrinkles, comprising administering a cosmetic composition to a subject in need thereof, wherein the cosmetic composition comprises the starfish-derived collagen peptide prepared by the method of claim 1.

11. A cosmetic composition comprising an elastic liposome comprising the starfish-derived collagen peptide prepared by the method of claim 1.

12. A method for improving skin wrinkles, comprising administering a cosmetic composition to a subject in need thereof, wherein the cosmetic composition comprises an elastic liposome comprising the starfish-derived collagen peptide prepared by the method of claim 1.

13. A method for anti-oxidation, comprising administering a cosmetic composition to a subject in need thereof, wherein the cosmetic composition comprises the starfish-derived collagen peptide prepared by the method of claim 1.

14. A method for anti-oxidation, comprising administering a cosmetic composition to a subject in need thereof, wherein the cosmetic composition comprises an elastic liposome comprising the starfish-derived collagen peptide prepared by the method of claim 1.
